(19) United States
(12) Patent Application Publication
(10) Pub. No.: US 2021/0322589 A1
(43) Pub. Date: Oct. 21, 2021

(54) Method for using Human-safe UVC to Control Contagion Presence within the Aircraft Cabin

(71) Applicant: Dennis Lynn Matter, Woodinville, WA (US)

(72) Inventor: Dennis Lynn Matter, Woodinville, WA (US)

(21) Appl. No.: 16/851,594

(22) Filed: Apr. 17, 2020

(51) Int. Cl. A61L 2/10; B64D 13/06; A61L 2/26; A61L 9/20

(52) U.S. Cl. CPC A61L 2/10; B64D 13/06; B64C 1/1423; A61L 9/20; A61L 2/26

(57) ABSTRACT

The present invention comprises a novel method for reducing the contribution of the aircraft toward the transport of human transmittable contagions, by location of anti-contagion apparatus at selected zones within the aircraft cabin and at key locations outside the aircraft. The anti-contagion apparatus being comprised of a source or array of sources of UVC operating in the human-safe range between 200 nm and 230 nm, and contained within a housing or fixture in a manner to directly expose humans standing within or passing through the space of coverage to a sufficient level of irradiation to kill exposed contagions on their person, in their breathed air, and on any surrounding touch surfaces. The invention encompasses the establishment of aircraft/airport related UVC₂₂₂ zones and stations for purpose sole purpose of contagion control, as well as certain design features of the UVC₂₂₂ apparatus generating the desired coverage volume and irradiation intensity required of the zone/station application.

### RELATED APPLICATIONS

None

### RELATED RESEARCH AND DEVELOPMENT

Federally Sponsored: None Other Jointly Sponsored: None Prior Disclosures: None

### FIELD OF INVENTION

The present invention relates to a novel method for reducing levels of harmful highly transmittable bacteria and viruses brought onto the aircraft, incubated in the in-flight cabin environment, then carried off the aircraft by the disembarking passengers and crew.

BACKGROUND

It is already established that UVC irradiation, when applied at sufficient levels, is an effective means of killing potentially-harmful highly-transmittable bacteria and viruses, referred to hereafter as contagions.

More recent research has begun to establish a ‘human-safe’ dividing line within the UVC band at around 230 nm wavelength, where the portion of the band between 200 nm and 230 nm is considered ‘safe’ for direct human exposure, while direct exposure to irradiation at wavelengths from 230 nm and longer is not considered to be safe.

Technological advancements have made possible the usage of high-intensity ultra-violet lamps operating in the narrow human-safe band between 200 nm and 230 nm. Furthermore, these lamps are able to deliver sufficient anti-contagion irradiation power to treat large spacial volumes.

The subject of this invention applies to direct application of the aforementioned human-safe anti-contagion UVC irradiation band between 200 nm to 230 nm wavelength, and centered at but not limited to 222 nm, henceforth referred to as UVC₂₂₂.

The subject of this invention applies to the provision of UVC₂₂₂ irradiation in selected common areas of the aircraft and associated service facilities, such as but not limited to, the aircraft cabin and passenger loading facility (otherwise known as the jetbridge or jetway), and doing so during periods where direct exposure of passengers and crew at anti-contagion power levels is intended by the invention.

The subject of this invention applies to the destruction of contagions residing within a volumetric space, hereafter referred to as a zone or station, where the contagions may be airborne, or existing on exposed areas of humans passing within the space, or on exposed physical surfaces (touch surfaces) within the space.

It is understood that the cabin of a civil aircraft often receives a large charge of potentially transmittable contagions brought onboard by passengers during aircraft loading.

It is further understood that the cabin of a civil aircraft acts as an incubator for contagions, potentially infecting any number of other passengers throughout the flight.

And it is further understood that during off-loading of passengers and crew, the contagion then spreads into the destination environment, thereby quickly jumping regions and continents in mostly transparent manner.

Finally, it is understood that cleaning a contaminated aircraft by use of potentially hazardous chemicals endangers cleaning personnel, results in a cocktail of contaminated materials that must be disposed of, and most often has already resulted in the contagion being allowed entry into one or more new regions before it is mitigated.

With the preceding understandings in mind, it is the objective of the invention described herein to apply a human-safe UVC₂₂₂ emitting apparatus for the sole purpose of reducing the amount of contagions brought onto the aircraft, then limiting the extent of person-to-person transmittal within the aircraft cabin during the flight, and thereby reducing the contagion load brought into the destination environment by disembarking passengers and crew.

The invention comprises the placement of UVC₂₂₂ anti-contagion apparatus at key ‘choke points’ in the movement of passengers and crew within the aircraft cabin and passenger service facilities. Choke points are considered to be places where passengers, crew and perhaps service personnel are moving through or standing within a common space, such as at an aircraft entry door, or outside an aircraft lavatory.

### BRIEF SUMMARY OF THE INVENTION

The present invention comprises the implementation of UVC₂₂₂ irradiation zones within public spaces and crew spaces, such as but not limited to, an aircraft cabin and passenger loading facility (jetbridge). The sole purpose of these UVC₂₂₂ zones is to substantially reduce the level of transmittable contagions, such as a virus or bacteria, carried onto the aircraft, transmitted to others during the flight, and then brought off the aircraft into the destination environment. The UVC₂₂₂ irradiated within these zones is specifically constrained to wavelengths between 200 nm and 230 nm, considered effective for killing human transmittable contagions, while also considered safe for direct human exposure. The invention encompasses the creation of anti-contagion zones that are generally of a sufficient size, shape, volume, and UVC₂₂₂ irradiation intensity to expose a person or persons passing through or standing within the zone with a wash of contagion killing irradiation being emitted from ceiling and/or wall mounted UVC₂₂₂ sourcing apparatus. The invention may be implemented as a singular UVC₂₂₂ emitting source (lamp or LED) or an array of sources as might be necessary to provide the desired zonal coverage and anti-contagion working efficiency. The primary intent of the invention is to provide a sufficiently broad wash of irradiation intensity for floor-to-ceiling wall-to-wall anti-contagion treatment of a designated public area (common space), but is equally applicable to treating more confining spaces, such as a lavatory or the food processing areas within the galley.

### BRIEF DESCRIPTION OF THE DRAWINGS:

Some embodiments of the present invention are illustrated as an example and are not limited by the figures of the accompanying drawings, in which the like references may indicate similar elements and in which:

FIG. 1-FIG. 1 illustrates one example, but not the only example, of (1) anti-contagion zone(s) located at common areas within an aircraft cabin (2) irradiating human-safe anti-contagion UVC at the 222 nm wavelength (3) onto passengers and crew entering the cabin via an opened access door, (4) onto passengers and crew outside lavatories or in other common standing areas, (5) onto passengers and crew using the lavatory facility, (6) onto crew and passengers present in the galley area, (7) onto specific food preparation actions taking place within the galley area.

FIG. 2-FIG. 2 illustrates one example, but not the only example, of (1) anti-contagion station(s) located along the jetbridge walkway or at other aircraft access point, (2) irradiating human-safe UVC at the 222 nm wavelength (3) onto passengers, crew and gate service personnel as they pass through the station(s). The anti-contagion station's irradiation source (2) might be part of a fixed or portable standalone station, or as panels or fixtures built into a jetbridge, galley service truck other aircraft servicing facility and (3) where the irradiating panels or fixtures are located in the ceiling and/or walls in a manner to best irradiate the intended area of coverage.

FIG. 3A-FIG. 3A illustrates an example, but not the only example, of how anti-contagion zones or stations can be configured to provide a volumetrically large area wash of UVC₂₂₂ irradiation being projected from the ceiling downward toward the floor. FIG. 3B-FIG. 3B illustrates a similar example, but not the only example, of the UVC₂₂₂ irradiation being projected from a side-wall outward across the horizontal space of the zone or station.

In both FIG. 3A and FIG. 3B embodiments, the basic irradiating element is (1) the UVC₂₂₂ lamp unit. One or more UVC₂₂₂ lamp units might be (3) arranged within a panel or fixture of various configurations as best suits the constraints of the installed space and the (4) desired coverage volume of coverage.

FIG. 4-FIG. 4 illustrates one example, but not the only example, of the present invention showing an anti-contagion lamp unit being comprised of (1) one or more UVC₂₂₂ lamps (2) mounted in a reflective housing assembly (3) optionally fitted with a cooling apparatus and filter assembly (4) a protective screen and bezel assembly and (5) being interconnected with and controlled by a lamp control and power supply assembly. The (1) lamp may be protected from dust accumulation by (3) forced air flow through an active or passive filter and/or (4) an active filter as part of the lamp protective screen. The (3) cooling and filter assembly may be fitted with (6) an additional indirect means of anti-contagion irradiation, for example by using low-cost LED emitters operating in the 250 nm wavelength to treat the airflow passing through the apparatus.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, the description will refrain from repeating every possible combination of the individual steps in an unnecessary fashion. Nevertheless, the specification and claims should be read with the understanding that such combinations are entirely within the scope of the invention and the claims.

Anti-contagion zones and stations, using irradiation apparatus operating in the human-safe UVC 200 nm to 230 nm wavelengths, and their associated lamp unit design features are discussed herein. In the following descriptions, for the purposes of explanation, numerous specific details are set forth in order to provide a thorough understanding of the present invention. It will be evident, however, to one skilled in the art that the present invention may be practiced without these specific details.

The present disclosure is to be considered an exemplification of the invention, and is not intended to limit the invention to the specific embodiments illustrated by the figures or descriptions below.

The following definitions are applied for better understanding of this discussion, but shall not be considered as constraining the invention to a specific application, design embodiment, or UVC lamp technology. Whereas a ‘zone’ is considered a free volume of UVC irradiation filling a space or volume of space without constraint of physical boundaries, a ‘station’ is considered to be the implementation of that zone within a defined set of physical boundaries. Whereas UVC defines a broad band of anti-contagion wavelengths existing between 200 nm and 300 nm, UVC₂₂₂ defines a human-safe subset of that band existing between 200 nm and 230 nm and centered at around 222 nm wavelength.

The present invention will now be described by referencing the appended figures, representing preferred embodiments.

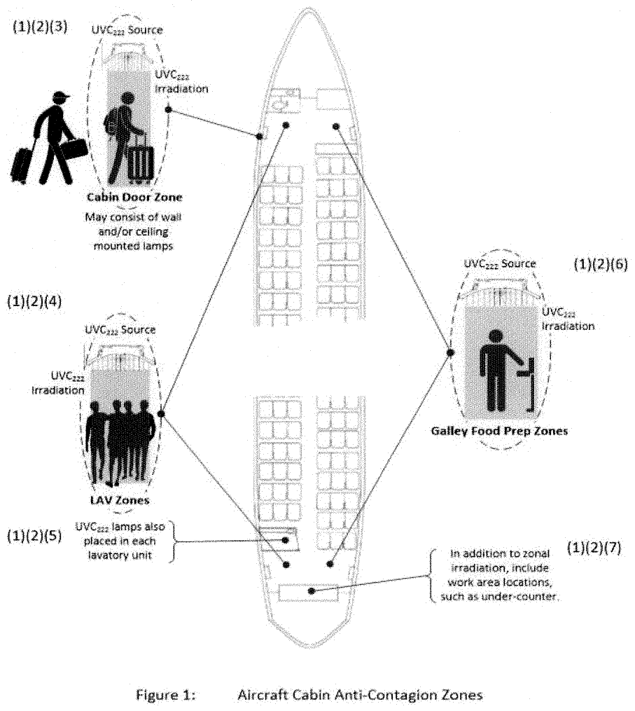

FIG. 1 describes the implementation of UVC₂₂₂ irradiated anti-contagion zones at key locations of the aircraft cabin as a means of reducing the level of unhealthy human transmitted bacteria and viruses within the aircraft cabin. These zone are thought to be best implemented at, but not limited to, cabin locations where passengers commonly pass through or stand within. Examples of such zones or locations may include (3) the aircraft cabin access doors, (4) the standing or waiting area outside the aircraft lavatories, (5) the interior of the aircraft lavatory, (6) the galley food preparation areas, and (7) work counters and other spaces within the galley.

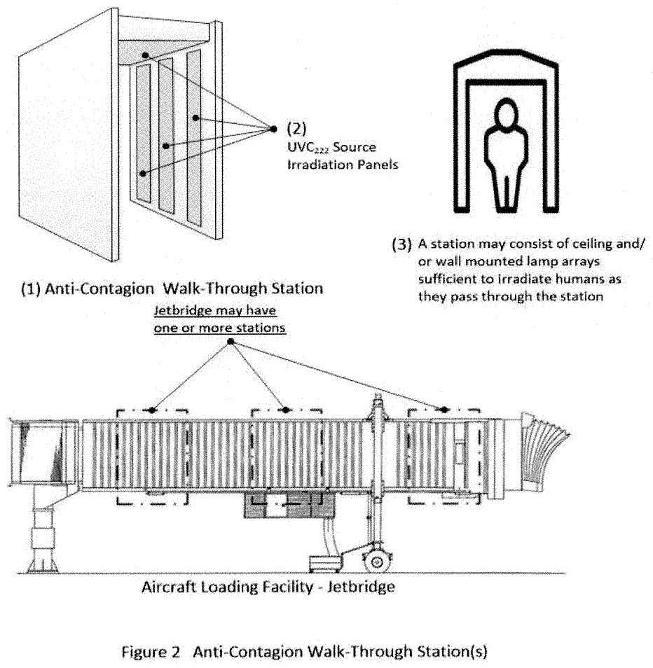

FIG. 2 describes the implementation of UVC₂₂₂ irradiated anti-contagion stations at key locations of the airport as a means of reducing the level of unhealthy human transmitted bacteria and viruses being brought into the aircraft during loading or from the aircraft into the airport during unloading. These (1)(2) stations are thought to be best implemented at, but not limited to, (1) aircraft service facilities where passengers and crew commonly (3) pass through when entering or exiting the aircraft. Similarly, (2) stations may be located at other natural control points within the airport, such as at security check points, along automated or non-automated walkways and at intra-airport transit stations, and within customs and immigration areas.

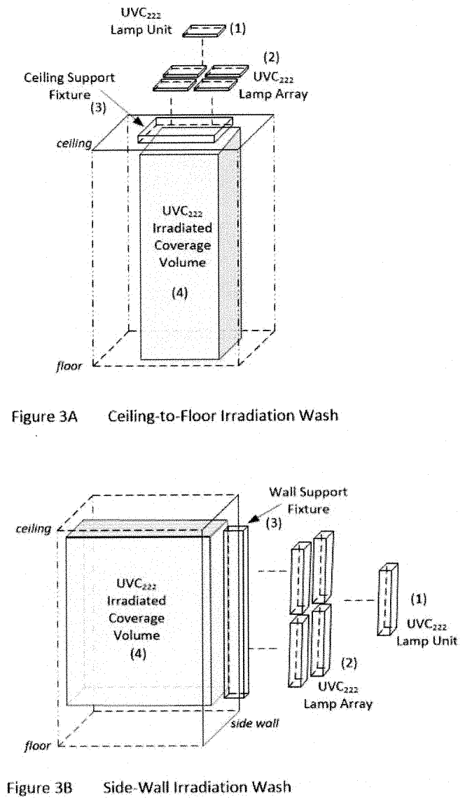

FIG. 3A and FIG. 3B describe the means, but not the only means, for applying the UVC₂₂₂ irradiation in a manner filling the volumetric space of a station or zone by locating (1) UVC₂₂₂ lamp unit or (2) array of such lamp units in a manner to form (3) a ceiling or wall mounted panel or fixture able to provide (4) uniform irradiation of humans and objects passing through or residing within the desired volume of coverage. It is understood by the invention that the (1) UVC₂₂₂ lamp or (2) array of lamps have been selected and configured to provide the necessary irradiation power and exposure envelope within the space for the sole purpose of killing contagions that might be exposed to the irradiation within that space, whether these contagions are present in the air, on exposed parts of a person's clothing or personal items or on surrounding touch surfaces that might be exposed to the irradiation.

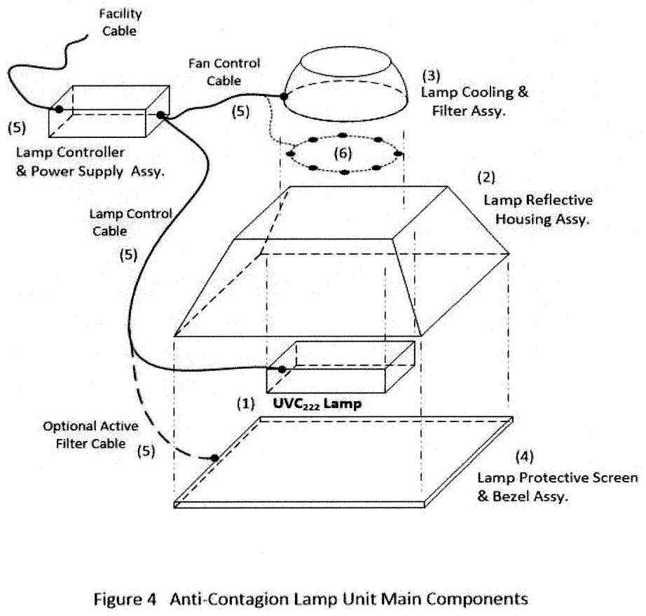

FIG. 4 describes one possible configuration of main components, but not the only possible configuration, making up a UVC₂₂₂ anti-contagion lamp unit or assembly. The lamp unit or assembly is envisioned to consist of (1) a UVC₂₂₂ lamp (2) a housing within which the lamp or array of lamps mount and which reflects the UVC₂₂₂ irradiation outward toward the intended anti-contagion space, and which also forms the mounting points for installation of the lamp unit into the intended application space, and (3) possibly including a lamp cooling and filter assembly attached to the housing for the purpose of keeping the lamp(s) within a desired temperature range and the irradiating surfaces of the lamp(s) clean of dust and particulates that might diminish the irradiation effectiveness of the lamp(s) and (4) possibly a lamp protective screen or grill and bezel assembly for the primary purpose of preventing unintended contact with the lamp's irradiation surfaces in a manner that efficiently passes the UVC₂₂₂ irradiation into the intended application space, and secondly an active or passive dust cleaning feature of the grill, and thirdly to provide the appearance finish for the intended application, and (5) a lamp controller and power supply assembly that firstly converts facility electrical power into the appropriate voltages and currents needed to operate the lamp(s) and any associated cooling and filtering devices, and secondly managing such functions as externally initiated on/off control and internally generated operational and safety features, and (6) a means of further clearing contagions by exposing air flowing through the enclosed (3) cooling and filtering assembly and (2) housing to the range of non-human-safe UVC.

What is claimed is:

1. The direct irradiation of human-safe UVC encompassing the band of 200 nm to 230 nm within occupied areas of the aircraft cabin for the express purposes of;
a. Reducing the amount of human transmitted contagions brought onto the aircraft from the outside by passengers and crew.
b. Reducing the role of the aircraft cabin as an incubator of contagions.
c. Reducing the amount of human transmittable contagions brought off the aircraft by disembarking passengers and crew at the destination.

2. The direct irradiation of human-safe UVC encompassing the band of 200 nm to 230 nm within certain occupied areas of the airport and aircraft service facilities as a means of further diminishing the role that passenger aircraft play in the global propagation of transmittable contagions.

3. The application of claim 1 human-safe anti-contagion zones at key areas of the aircraft cabin where passengers and crew commonly pass through or collect, including but not limited to;
a. The aircraft primary access doors.
b. Standing areas outside of lavatories.
c. Lavatory interiors.
d. Galley food preparation areas.

4. The application of claim 2 human-safe anti-contagion stations at key areas of the airport where passengers, crews and service personnel pass through, including but not limited to;
a. The aircraft passenger loading facilities (jetbridge).
b. The aircraft galley services facilities.
c. Other common choke points, such as security checks and transport systems.

5. The nature and characteristics of claim 3 and claim 4 as comprising all manner of form and future deemed appropriate to achieve an optimum level of claim 1 and claim 2 anti-contagion irradiation within the designated zone or station, including but not limited to;
a. irradiation power density (sufficient to kill contagions within the zone volume).
b. geometry (a consideration of even coverage within the allocated zone).
c. method (ceiling or wall mounted irradiation sources, or both).
d. integrations (whether standalone or integrated into other systems or equipment).
e. restrictions (any regulatory considerations of limits on human exposure).

6. Key features of the irradiation source of claim 5, including but not limited to;
a. A source (lamp) of UVC operating at claim 1 wavelength and capable of the claim 5 coverage and power characteristics as a single item or an array, b. An enclosure or panel and finish covers housing the irradiation source(s). c. A means of maintaining the source (lamp) within optimum ranges. d. An active or passive air filtering method to control dust accumulation. e. A human safe internal source of 250 nm UVC contained within the air flow path. f. An efficient active or passive dust filtering method within the irradiation path.
